(19) United States
(12) Patent Application Publication
(10) Pub. No.: US 2022/0101996 A1
(43) Pub. Date: Mar. 31, 2022

(54) HEALTH CONDITION DETECTION AND MONITORING USING ARTIFICIAL INTELLIGENCE

(71) Applicant: KYNDRYL, INC., NEW YORK, NY (US)

(72) Inventors: Claudio Keiji Iwata, Sao Paulo (BR); Sergio Varga, Campinas (BR); Isabel Carolina Zorzi de Miranda, Sao Paulo (BR); Maria Clara Verçosa da Silva Rodrigues, Rio de Janeiro (BR); Marcia Ito, Sao Paulo (BR)

(21) Appl. No.: 17/039,212

(22) Filed: Sep. 30, 2020

(51) Int. Cl. G16H 50/20; G16H 80/00; G16H 30/40

(52) U.S. Cl. CPC G16H 50/20; G16H 30/40; G16H 80/00

(57) ABSTRACT

Detection and monitoring of health conditions that are accompanied by an injury site includes receiving, by one or more processors, an identifier associated with a patient and an associated first image, the first image corresponding to an injury in the injury site. The one or more processors analyze the associated first image using a machine learning model that includes an inference engine and a knowledge base. The machine learning model compares the associated first image against a patient data history and a tracking database. In response to a determination the injury site is associated with the health condition, the one or more processors generate a first result including sending a report containing the first result to a healthcare professional caring for the patient and display the first result informing the patient and healthcare professional the injury needs special care including requiring an appointment.

### BACKGROUND

The present invention generally relates to the field of artificial intelligence, and more particularly to a method, system and computer program product for detecting and monitoring health conditions accompanied by an injury site.

Certain health conditions can cause skin lesions such as ulcers, rashes, sores, and the like. In some cases, these skin lesions can deteriorate quickly without proper medical attention. For instance, diabetic foot, a long-term or chronic complication of diabetes that results directly from peripheral arterial disease (PAD) and/or sensory neuropathy affecting the feet of diabetic patients, is associated with a diabetic foot ulcer. A diabetic foot ulcer is an open sore or wound that can occur in patients with diabetes, and is commonly located on the bottom of the foot. Treatment of diabetic foot complications accounts for 15-25% of total healthcare resources for diabetes. It is estimated that with basic diabetes management care, up to 80% of all diabetic foot amputations can be prevented. However, lack of patient engagement may occur due to problems of health literacy, decision-making delays during treatment, and inadequate self-management of chronic conditions.

### SUMMARY

A possible reason for lack of patient engagement in self-management of chronic health conditions such as diabetes may be insufficient communication between patients and caregivers. Engaging patients in their health care routine and encouraging them to take responsibility for their own health may be the best way to improve people's health and ensure the sustainability of health systems. Frequently, patients look for medical treatment of injuries when it is too late. In the case of diabetic patients, for example, an uncontrolled infection on an injury site may cause the amputation of the affected member. This type of situation may be avoided with periodic evaluations (e.g., daily) of injury sites by a medical professional.

The present disclosure recognizes the shortcomings and problems associated with managing health conditions that are accompanied by an injury site. Particularly, the need for an automated and efficient method to conduct daily self-check of an existing injury by patients that can also provide real-time feedback to caregivers regarding changes in the monitored area to potentially prevent or help diagnose health conditions associated with such injury. Therefore, there is a need for a method and system for monitoring and detecting health conditions accompanied by an injury site that facilitates patients, health care professionals, and health authorities to manage and prevent complications associated with the health condition.

Shortcomings of the prior art are overcome and additional advantages are provided through the provision of a computer-implemented method for detecting and monitoring a health condition accompanied by an injury site that includes receiving, by one or more processors, an identifier associated with a patient and an associated first image, the first image corresponding to an injury in the injury site. The one or more processors analyze the associated first image using a machine learning model that includes an inference engine and a knowledge base. The machine learning model compares the associated first image against a patient data history and a tracking database. In response to a determination the wound is associated with the health condition, the one or more processors generate a first result including sending a report containing the first result to a healthcare professional caring for the patient and display the first result informing the patient and healthcare professional the injury needs special care including requiring an appointment.

Another embodiment of the present disclosure provides a computer program product for detecting and monitoring a health condition accompanied by an injury site, based on the method described above.

Another embodiment of the present disclosure provides a computer system for detecting and monitoring a health condition accompanied by an injury site, based on the method described above.

### BRIEF DESCRIPTION OF THE DRAWINGS

The following detailed description, given by way of example and not intended to limit the invention solely thereto, will best be appreciated in conjunction with the accompanying drawings, in which:

FIG. 1 is a block diagram illustrating a networked computer environment, according to an embodiment of the present disclosure;

FIG. 2 depicts a functional flow diagram including components of a system for detection and monitoring of a health condition accompanied by an injury site using artificial intelligence, according to an embodiment of the present disclosure;

FIGS. 3A-3B depict a flowchart illustrating the steps of a computer-implemented method for detection and monitoring of a health condition accompanied by an injury site using artificial intelligence, according to an embodiment of the present disclosure;

FIG. 4 is a block diagram of internal and external components of a computer system, according to an embodiment of the present disclosure;

FIG. 5 is a block diagram of an illustrative cloud computing environment, according to an embodiment of the present disclosure; and

FIG. 6 is a block diagram of functional layers of the illustrative cloud computing environment of FIG. 5, according to an embodiment of the present disclosure.

The drawings are not necessarily to scale. The drawings are merely schematic representations, not intended to portray specific parameters of the invention. The drawings are intended to depict only typical embodiments of the invention. In the drawings, like numbering represents like elements.

### DETAILED DESCRIPTION

Detailed embodiments of the claimed structures and methods are disclosed herein; however, it can be understood that the disclosed embodiments are merely illustrative of the claimed structures and methods that may be embodied in various forms. This invention may, however, be embodied in many different forms and should not be construed as limited to the exemplary embodiments set forth herein. In the description, details of well-known features and techniques may be omitted to avoid unnecessarily obscuring the presented embodiments.

Embodiments of the present invention provide a method, system, and computer program product for detecting and monitoring a health condition accompanied by an injury site. The following described exemplary embodiments provide a system, method, and computer program product to, among other things, monitor a patient's injury by implementing machine learning techniques to analyze images provided by the patient (e.g., from cellphones, tablets, and the like), compare the images against a personalized health database, and based on the comparison and a health history of the patient, automatically generate and send a report containing a result of the comparison to the patient's caregivers.

Thus, the present embodiments have the capacity to improve the technical field of artificial intelligence by creating a database of historic images depicting the evolution of an injury site, maintaining efficient communication between patient and health care professionals, integrating patients, doctors, hospitals, and/or health authorities to provide a solution for monitoring and controlling injuries to prevent complications associated with certain health conditions. Moreover, the use of artificial intelligence to evaluate the images allows the creation of a centralized database including updates on patient's profile, medical history, and new technologies that can be used for evaluation of patient's test/exams. This in turn may provide doctors and caregivers with a complete picture of a patient's health history, facilitating accurate and faster diagnosis. By preventing health conditions that can be accompanied by an injury site, such as diabetic foot, and its complications including member amputation, embodiments of the present disclosure may help reduce doctor visits, surgeries, hospitalizations and associated costs.

Additionally, the present embodiments can generate a patient profile from the analysis of different data sources using big data analytics and machine learning techniques. The different data sources used by the present embodiments may include annotated dialog corpus (composed by previous dialogs between the patients/caregivers and the care team), patient-generated health data, and patient clinical data. This historic clinical profile along with additional information provided by the patient on a regular basis (e.g., daily, weekly, etc.) can enable classifying patients according to a stage of the disease and help identifying the best treatment approach. The proposed self-check strategy provides patients and health professionals with insights on the evolution of injuries, tips to prevent and control infections, diagnose possible problems, and/or identify when the patient(s) needs to anticipate or postpone a doctor appointment. Further, the proposed embodiments, provide a way to improve health information exchange between patient and caregivers, and between care team and care coordinators/managers in order to keep patients informed about their current medical condition(s).

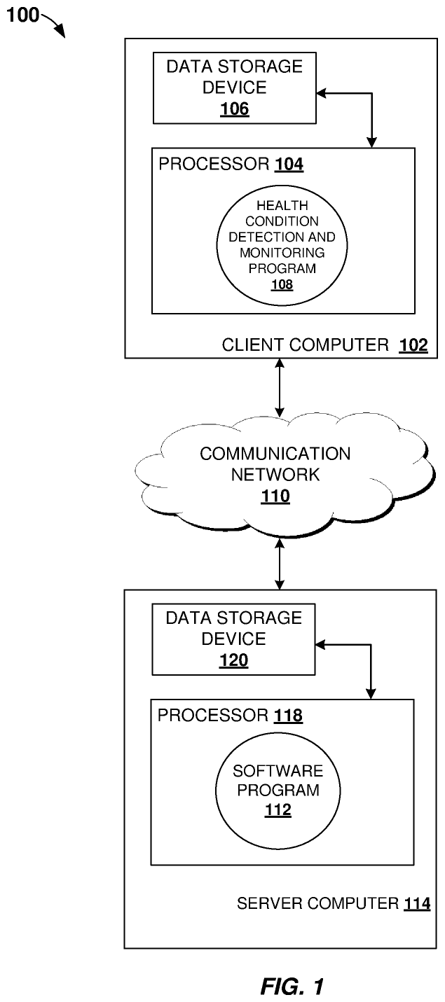

Referring now to FIG. 1, an exemplary networked computer environment 100 is depicted, according to an embodiment of the present disclosure. FIG. 1 provides only an illustration of one embodiment and does not imply any limitations with regard to the environments in which different embodiments may be implemented. Many modifications to the depicted environment may be made by those skilled in the art without departing from the scope of the invention, as recited by the claims.

The networked computer environment 100 may include a client computer 102 and a communication network 110. The client computer 102 may include a processor 104, that is enabled to run a health condition detection and monitoring program 108, and a data storage device 106. Client computer 102 may be, for example, a mobile device, a telephone (including smartphones), a personal digital assistant, a netbook, a laptop computer, a tablet computer, a desktop computer, or any type of computing devices capable of accessing a network.

The networked computer environment 100 may also include a server computer 114 with a processor 118, that is enabled to run a software program 112, and a data storage device 120. In some embodiments, server computer 114 may be a resource management server, a web server or any other electronic device capable of receiving and sending data. In another embodiment, server computer 114 may represent a server computing system utilizing multiple computers as a server system, such as in a cloud computing environment.

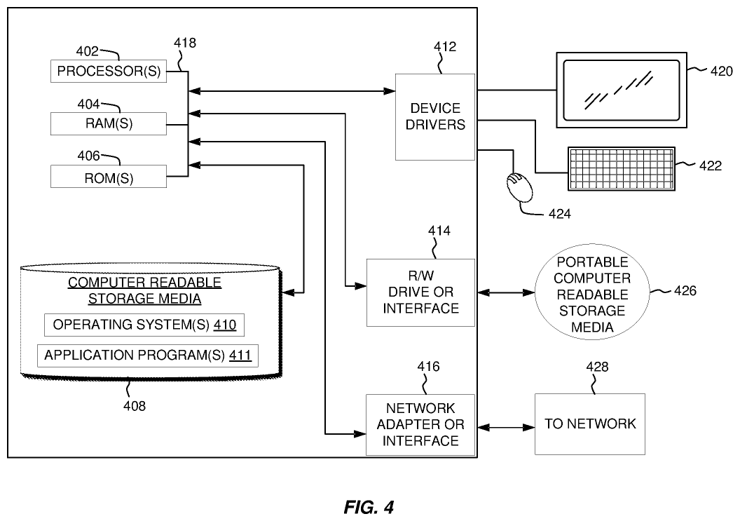

The health condition detection and monitoring program 108 running on client computer 102 may communicate with the software program 112 running on server computer 114 via the communication network 110. As will be discussed with reference to FIG. 4, client computer 102 and server computer 114 may include internal components and external components.

The networked computer environment 100 may include a plurality of client computers 102 and server computers 114, only one of which is shown. The communication network 110 may include various types of communication networks, such as a local area network (LAN), a wide area network (WAN), such as the Internet, the public switched telephone network (PSTN), a cellular or mobile data network (e.g., wireless Internet provided by a third or fourth generation of mobile phone mobile communication), a private branch exchange (PBX), any combination thereof, or any combination of connections and protocols that will support communications between client computer 102 and server computer 114, in accordance with embodiments of the present disclosure. The communication network 110 may include wired, wireless or fiber optic connections. As known by those skilled in the art, the networked computer environment 100 may include additional computing devices, servers or other devices not shown.

Plural instances may be provided for components, operations, or structures described herein as a single instance. Boundaries between various components, operations, and data stores are somewhat arbitrary, and particular operations are illustrated in the context of specific illustrative configurations. Other allocations of functionality are envisioned and may fall within the scope of the present invention. In general, structures and functionality presented as separate components in the exemplary configurations may be implemented as a combined structure or component. Similarly, structures and functionality presented as a single component may be implemented as separate components. These and other variations, modifications, additions, and improvements may fall within the scope of the present invention.

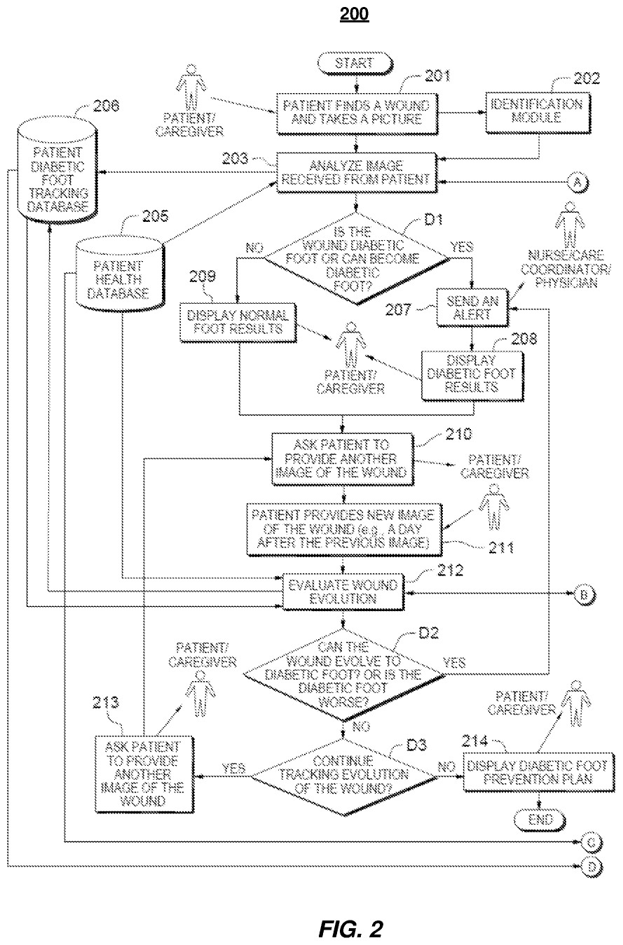

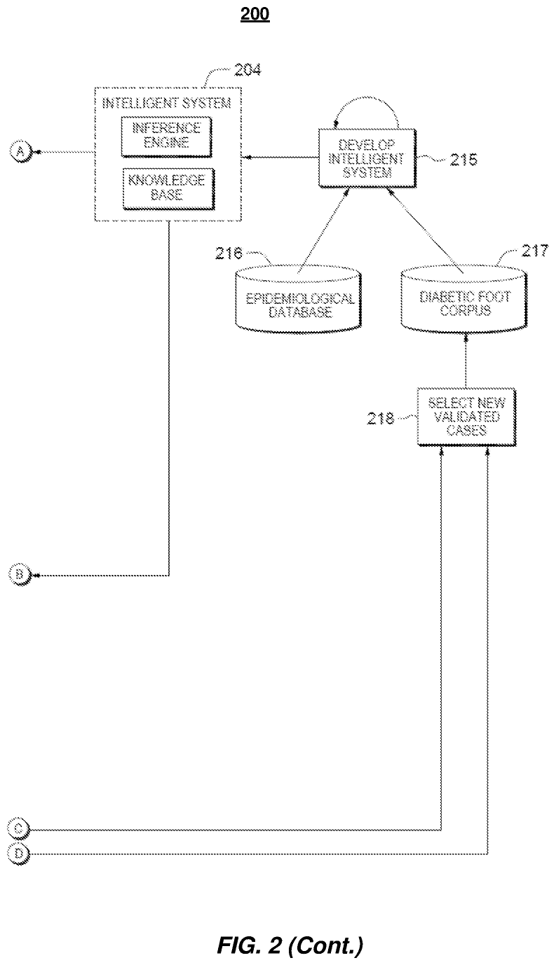

Referring now to FIG. 2, a functional flow diagram depicting components of a system 200 for detection and monitoring of health conditions accompanied by an injury site is shown, according to an embodiment of the present disclosure. In this embodiment, at 201, a patient finds or notices an injury in a body part (hereinafter “injury site”). The injury in the injury site may include, for example, any type of skin lesion or an area that looks different from the surroundings. Typical skin lesions that can be associated with the injury may include any type of wounds, ulcers, rashes, sores, blisters, and the like. Particularly, the system 200 can track any visible change in the patient's body.

For illustration purposes only, without intent of limitation, operation of the system 200 will be explained using as example a patient with an injury site including a wound that could be associated with diabetic foot. In this example embodiment, the patient proceeds to take a picture using any available electronic device furbished with a camera and capable of establishing a network connection. For example, the patient takes the picture using a smartphone or tablet device. Subsequently, the patient sends or provides these images to the proposed system 200 for detection and monitoring of health conditions accompanied by an injury site, as will be described in detail below.

The images are received at 202 by an identification module in which each image is associated with a patient identification number (ID). Subsequently at 203, an analysis module analyzes the received images using an intelligent system 204 that is capable of recognizing the wound and determining whether the wound is a diabetic foot or not. If the wound is not a diabetic foot, the intelligent system 204 can predict whether the wound can become a diabetic foot. The intelligent system 204 uses pictures, patient's clinical data (e.g., type of diabetes, blood glucose result, glycated hemoglobin result, etc.), and patient-generated health data (e.g., daily blood glucose measurements, calories ingested, mood, amount of physical exercise done, etc.).

The intelligent system 204 is the machine learning trained portion of the system for detection and monitoring diabetic foot of FIG. 2. The intelligent system 204 further includes an inference engine and a knowledge base capable of analyzing the received images and evaluating whether the wound can be classified as diabetic foot or not. The intelligent system 204 is also capable of predicting a probability of the wound becoming diabetic foot. The inference engine of the intelligent system 204 is based on machine learning algorithms, ontology, knowledge graph, rules, case-based reasoning, and the like. According to an embodiment, the intelligent system 204 generates a report including the analysis performed on the received images.

Patient's health data can be obtained from a patient health database 205 that contains clinical data such as, for example, patient structured data from electronic medical records (EMR), electronic health records (EHR), and/or hospital information systems (HIS). The patient health database 205 may also contain patient-generated health data including, for example, health-related data created, recorded, or gathered by patients, family members, and/or caregivers to help address a health concern. The patient-generated health data may include, but is not limited to, a health history of the patient, a treatment history, biometric data, symptoms, and lifestyle choices. The patient-generated health data is different from data generated in clinical settings and through encounters with providers in two important ways: (1) patients, not providers, are primarily responsible for capturing or recording the data, and (2) patients decide how to share or distribute the data to health care providers and other entities. According to an embodiment, the system of FIG. 2 uses anonymized health data. Data anonymization is the process of protecting private or sensitive information by erasing or encrypting identifiers that connect an individual to stored data.

It should be noted that any user (i.e., patient) data collection (e.g., patient's clinical data, patient-generated health data, etc.) by embodiments of the present disclosure is done with user consent via an opt-in and opt-out feature. As known by those skilled in the art, an opt-in and opt-out feature generally relates to methods by which the user can modify a participating status (i.e., accept or reject the data collection). In some embodiments, the opt-in and opt-out feature can include a software application(s) available in, for example, client computer 102. Additionally, the user can choose to stop having his/her information being collected or used. In some embodiments, the user can be notified each time data is being collected. The collected data is envisioned to be secured and not shared with anyone without user's consent. The user can stop the data collection at any time.

The analysis module (203) stores results generated by the intelligent system 204 in a diabetic foot tracking database 206 associated with the patient. The stored results include patient ID, received images, timestamp (i.e., date and time of the day) of each received image, and the report of the analysis conducted by the intelligent system 204. According to an embodiment, it is important to store the generated results so they can be later used as input data for training the intelligent system 204. Additionally, stored results provide a baseline for monitoring the wound or diabetic foot over time. Stated differently, the diabetic foot tracking database 206 provides a history of the patient's wound that allows improving an accuracy of the intelligent system 204 and the monitoring (e.g., improvement or worsening) of the patient's wound or diabetic foot.

As mentioned above, the present embodiments can be implemented for monitoring and evaluation of health conditions accompanied by an injury site. However, it should be noted that a corpus, such as diabetic foot corpus 217, must be created for each specific health condition and used to train the intelligent system 204 for recognizing and monitoring the type of injury associated with each specific health condition. Other health conditions accompanied by an injury site may include, for example, psoriasis, skin cancer, vitiligo, etc. In these cases the system 200 can track the evolution of moles and/or skin areas that are unusual in color, size, shape or texture.

Based on the results generated by the intelligent system 204, if it is determined (D1) that the wound is or can become diabetic foot, the system 200 sends an alert at 207 including the report generated by the intelligent system 204 to the patient's caregivers (e.g., nurse, physician, or care coordinator). Then, the system 200 displays (208) a notification including a result of the analysis to both patient and caregivers alerting them that the wound may need special care and a follow-up appointment is recommended. In an embodiment, if it is determined (D1) that the wound is not at risk of becoming diabetic foot, the system 200 displays a notification to the patient (209) informing that the wound is not diabetic foot. The notification may also include a reminder to the patient to maintain appropriate wound care and blood sugar levels under control.

The system may ask the patient and/or caregiver on the following days (210) to provide another image of the wound to assess its evolution. The patient may provide another image of the wound (211) to the system of FIG. 2 for analysis. The system receives the new image of the wound (212) and compares it with previously received images to determine a current state of the wound. Specifically, the intelligent system 204 determines whether changes have occurred in the wound by comparing the new image against the stored images. The intelligent system 204 performs the process described above to determine whether a current state of the wound associated with the new image is or can become diabetic foot. If the system 200 determines (D2) that the wound is not diabetic foot, but there are changes in the new image, the intelligent system 204 can predict whether the wound can become diabetic foot. The intelligent system 204 uses new image(s), previously stored images (e.g., from previous day), patient's clinical data, and patient-generated health data to conduct one more time the analysis explained above. Results from the intelligent system 204 are stored in the diabetic foot tracking database 206.

If it is determined (D2) that the wound is not at risk of becoming diabetic foot (no worsening from previous record), the system 200 generates and displays a notification to the patient (209) informing that the wound is not a diabetic foot. The system 200 may prompt the patient (D3) to decide whether he/she desires continuing monitoring the wound. If the patients decides to continue monitoring the wound, the system 200 generates (213) a notification for the patient and/or caregiver to continue treating the wound, keeping blood sugar levels under control, and attending or scheduling follow-up appointments. The system 200 continues monitoring the wound (210) until the patients decides to stop the process (or opts out).

If the patient decides not to continue monitoring the wound, the system 200 generates and displays (214) a report including results of the analysis, diabetic foot prevention tips, and reminders of the importance of maintaining normal glucose levels and attending follow-up appointments with caregivers.

According to an embodiment, the inference engine and knowledge base of the intelligent system 214 can be developed by machine learning training or modeling using a diabetic foot corpus 217 (including ontology, knowledge graph, case-based reasoning, rules, etc.) combined with an epidemiological database 216. The diabetic foot corpus 217 includes a database storing (anonymized) data from a plurality of patients, the diabetic foot corpus 217 is necessary to create the intelligent system 204. The diabetic foot corpus 217 stores images, analysis reports, and any relevant data from the patient health database 205. The epidemiological database 216 includes different epidemiological data that can help creating an accurate predictive model for the intelligent system 204. Examples of epidemiological data may include, but are not limited to, patient's demographic information, previous injuries, infectious/non-infectious diseases, geographic location, and environmental exposure.

In some embodiments, relevant cases can be selected (e.g., from the patient health database 205, or the diabetic foot tracking database 206) to retrain the intelligent system 204. It should be noted that cases composing the diabetic foot corpus 217 originate from patients that, prior authorization, had their data collected and validated by the proposed system 200.

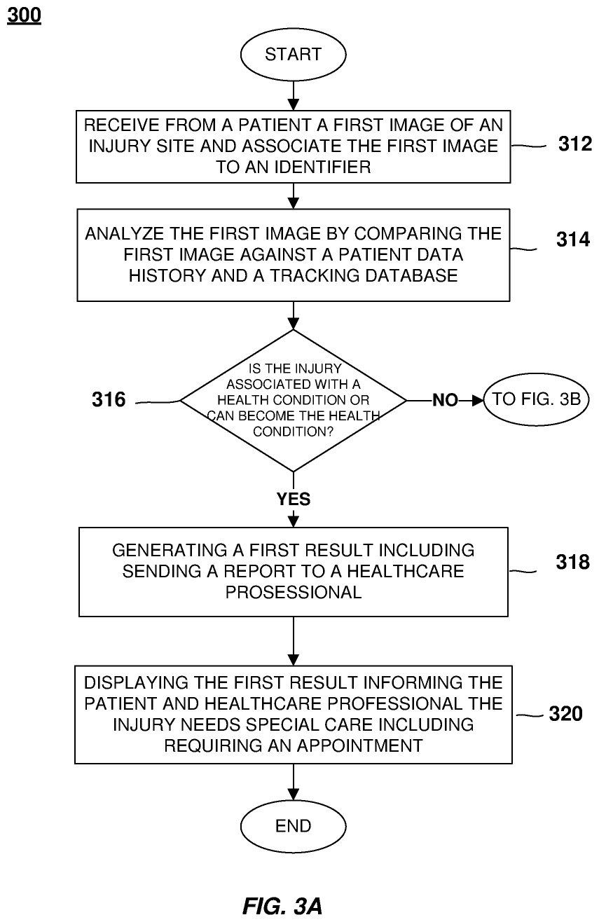

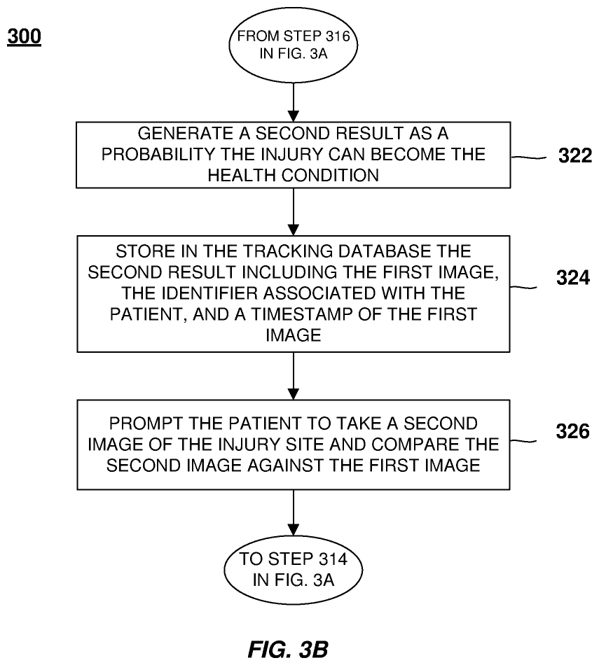

Referring now to FIGS. 3A-3B, a flowchart 300 illustrating the steps of a computer-implemented method for detection and monitoring of diabetic foot using artificial intelligence is shown, according to an embodiment of the present disclosure.

The process starts at step 312 by receiving from a patient a first image of an injury located on an injury site. The image is associated with an identifier (e.g., patient identification number) corresponding to the patient. At 314, the first image is analyzed by comparing the first image against a history of health data associated with the patient (i.e., patient health database 205 of FIG. 2) and a tracking database (i.e., patient diabetic foot tracking database 206 of FIG. 2) using the intelligent system 204 of FIG. 2.

In response to a determination the injury is associated with a health condition (e.g., diabetic foot, or any other skin lesion) at step 316, the method continues with step 318 by generating a first result including sending a report containing the first result to a healthcare professional caring for the patient. At step 320, the first result is displayed informing patient and healthcare professionals the injury site needs special care including requiring an appointment.

In response to a determination the injury is not associated with the health condition at step 316, a second result is generated at step 322, the second result includes a probability the injury site can become or be associated with the health condition. At step 324, the second result is stored in a tracking data base such as the patient diabetic foot tracking database 206 of FIG. 2. The second result further includes the identifier associated with the patient, a timestamp of the first image, the first image, and a report including the analysis performed by the intelligent system 204 of FIG. 2. The second result can further include generating and displaying an alert including a normal result for the health condition to the patient and/or caregivers. Additionally, in some embodiments, a reminder for the patient to maintain blood parameters associated with the health condition under control (e.g., normal blood glucose levels) and attend follow-up appointments can be generated.

At step 326, the patient is prompted to provide a second image of the injury site during the following days (e.g., the next day after providing the first image) for tracking and evaluation. The second image of the injury site is then compared against the associated first image to determine whether a change occurred or the injury can be associated with the health condition. At this step (326) the process returns to step 314 in FIG. 3A in which the intelligent system 204 of FIG. 2 conducts the health condition analysis on the received second image to determine whether the injury is better or worse (i.e., the probability of becoming the health condition has decreased or increased).

If, after the analysis, the second image still shows that the injury site is not associated with the health condition, a third result is generated, as a probability of the injury site becoming or being associated with the health condition. The third result is stored in the tracking database (e.g., the patient diabetic foot tracking database 206 of FIG. 2). The third result can be displayed together with reminders for the patient to continue appropriate care of the injury site and to maintain normal blood parameters (e.g., glucose levels). Further, health condition prevention results informing the patient and healthcare professional about the success of the treatment including a reminder of current treatment, importance of maintaining blood work levels associated with the health condition within a normal range, and returning for medical appointments on scheduled dates can be displayed to the patient and patient's caregivers.

Therefore, embodiments of the present disclosure provide a method, system and computer program product to, among other things, evaluating a state of an injury using machine learning techniques and leveraging patient's data to determine a probability of the injury being associated with certain health conditions. By using the proposed embodiments, the evolution of an injury site can be monitored by the patient in a non-controlled environment using common devices such as smartphones. Further, the proposed embodiments can generate alerts to patients and/or caregivers/health professionals when there is a higher probability of the injury being associated with the health condition. According to an embodiment, monitoring of the injury site can continue until the end of treatment or until the injury has healed.

Referring now to FIG. 4, a block diagram of components of client computer 102 and server computer 114 of networked computer environment 100 of FIG. 1 is shown, according to an embodiment of the present disclosure. It should be appreciated that FIG. 4 provides only an illustration of one implementation and does not imply any limitations regarding the environments in which different embodiments may be implemented. Many modifications to the depicted environment may be made.

Client computer 102 and server computer 114 may include one or more processors 402, one or more computer-readable RAMs 404, one or more computer-readable ROMs 406, one or more computer readable storage media 408, device drivers 412, read/write drive or interface 414, network adapter or interface 416, all interconnected over a communications fabric 418. Communications fabric 418 may be implemented with any architecture designed for passing data and/or control information between processors (such as microprocessors, communications and network processors, etc.), system memory, peripheral devices, and any other hardware components within a system.

One or more operating systems 410, and one or more application programs 411 are stored on one or more of the computer readable storage media 408 for execution by one or more of the processors 402 via one or more of the respective RAMs 404 (which typically include cache memory). In the illustrated embodiment, each of the computer readable storage media 408 may be a magnetic disk storage device of an internal hard drive, CD-ROM, DVD, memory stick, magnetic tape, magnetic disk, optical disk, a semiconductor storage device such as RAM, ROM, EPROM, flash memory or any other computer-readable tangible storage device that can store a computer program and digital information.

Client computer 102 and server computer 114 may also include a R/W drive or interface 414 to read from and write to one or more portable computer readable storage media 426. Application programs 411 on client computer 102 and server computer 114 may be stored on one or more of the portable computer readable storage media 426, read via the respective R/W drive or interface 414 and loaded into the respective computer readable storage media 408.

Client computer 102 and server computer 114 may also include a network adapter or interface 416, such as a TCP/IP adapter card or wireless communication adapter (such as a 4G wireless communication adapter using OFDMA technology) for connection to a network 428. Application programs 411 on client computer 102 and server computer 114 may be downloaded to the computing device from an external computer or external storage device via a network (for example, the Internet, a local area network or other wide area network or wireless network) and network adapter or interface 416. From the network adapter or interface 416, the programs may be loaded onto computer readable storage media 408. The network may comprise copper wires, optical fibers, wireless transmission, routers, firewalls, switches, gateway computers and/or edge servers.

Client computer 102 and server computer 114 may also include a display screen 420, a keyboard or keypad 422, and a computer mouse or touchpad 424. Device drivers 412 interface to display screen 420 for imaging, to keyboard or keypad 422, to computer mouse or touchpad 424, and/or to display screen 420 for pressure sensing of alphanumeric character entry and user selections. The device drivers 412, R/W drive or interface 414 and network adapter or interface 416 may include hardware and software (stored on computer readable storage media 408 and/or ROM 406).

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as Follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as Follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as Follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

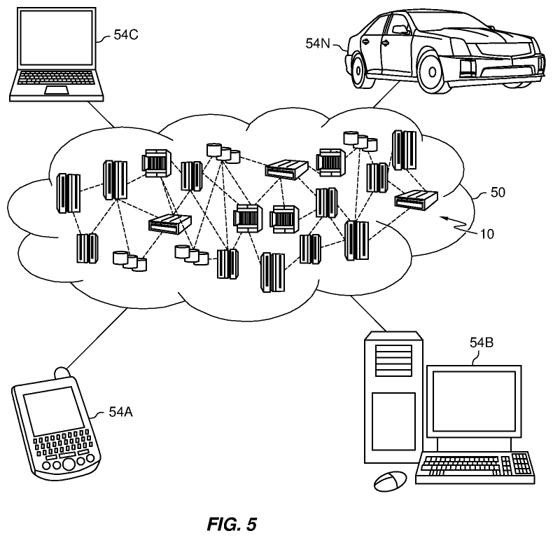

Referring now to FIG. 5, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 5 are intended to be illustrative only and that computing nodes 10 and cloud computing environment 50 can communicate with any type of computerized device over any type of network and/or network addressable connection (e.g., using a web browser).

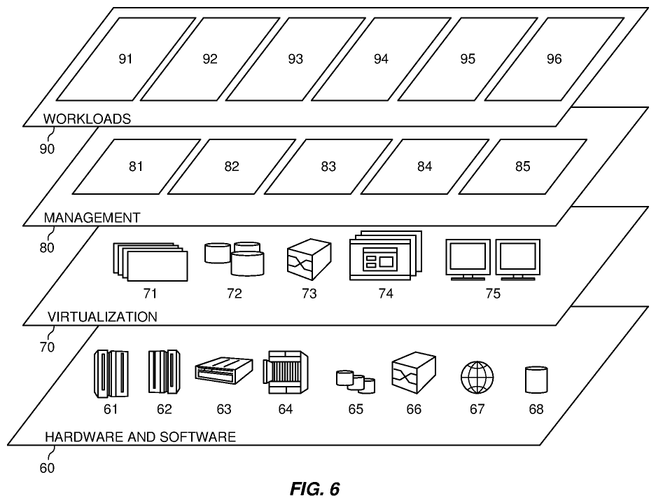

Referring now to FIG. 6, a set of functional abstraction layers provided by cloud computing environment 50 (FIG. 5) is shown. It should be understood in advance that the components, layers, and functions shown in FIG. 6 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and system for detection and monitoring of health conditions accompanied by an injury site 96.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While steps of the disclosed method and components of the disclosed systems and environments have been sequentially or serially identified using numbers and letters, such numbering or lettering is not an indication that such steps must be performed in the order recited, and is merely provided to facilitate clear referencing of the method's steps. Furthermore, steps of the method may be performed in parallel to perform their described functionality.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method for detecting and monitoring a health condition accompanied by an injury site, comprising:
receiving, by one or more processors, an identifier associated with a patient and an associated first image, the first image corresponding to an injury in the injury site in the patient's body;
analyzing, by the one or more processors, the associated first image using a machine learning model comprising an inference engine and a knowledge base, the machine learning model compares the associated first image against a patient data history and a tracking database;
in response to a determination the injury site is associated with the health condition, generating, by the one or more processors, a first result including sending a report containing the first result to a healthcare professional caring for the patient; and
displaying, by the one or more processors, the first result informing the patient and healthcare professional the injury site needs special care including requiring an appointment.

2. The method of claim 1, wherein the health condition comprises a diabetic foot and the patient data history comprises type of diabetes, blood glucose result, glycated hemoglobin result, and patient generated health data including daily blood glucose measurements, calories ingested, mood, and an amount of physical exercises done.

3. The method of claim 1, further comprising:
in response to a determination the injury site is not associated with the health condition, generating, by the one or more processors, a second result as a probability the injury site is associated with the health condition.

4. The method of claim 3, further comprising:
storing, by the one or more processors, the second result including the identifier associated with the patient, a date and time of the associated first image, the associated first image, and a report of an analysis in the tracking database.

5. The method of claim 3, further comprising:
in response to the second result displaying a normal result, generating, by the one or more processors, an alert comprising a reminder for the patient to maintain blood work levels associated with the health condition within a normal range.

6. The method of claim 1, further comprising:
prompting, by the one or more processors, the patient to take a second image of the injury site in a next day for tracking and evaluation;
in response to receiving the second image of the injury site, comparing, by the one or more processors, the second image with the associated first image using the machine learning model to determine whether a change occurred including whether the injury site is associated with the health condition; and
in response to a determination the injury site is not associated with the health condition while the second image includes changes, generating, by the one or more processors, a third result as a probability the injury site is associated with the health condition.

7. The method of claim 6, further comprising:
storing, by the one or more processors, the third result including updated clinical data of the patient and updated patient generated health data in the tracking database;
in response to the third result, displaying, by the one or more processors, a normal result comprising a current treatment and a reminder to maintain normal blood work levels; and
in response to a determination the wound is treated successfully, displaying, by the one or more processors, a health condition prevention result informing the patient and healthcare professional of a successful treatment including importance of maintaining normal blood work levels and returning for a medical appointment on a scheduled date.

8. A computer system for detecting and monitoring a health condition accompanied by an injury site, comprising:
one or more processors, one or more computer-readable memories, one or more computer-readable tangible storage devices, and program instructions stored on at least one of the one or more storage devices for execution by at least one of the one or more processors via at least one of the one or more memories, wherein the computer system is capable of performing a method comprising:
receiving, by one or more processors, an identifier associated with a patient and an associated first image, the first image corresponding to an injury in the injury site in the patient's body;
analyzing, by the one or more processors, the associated first image using a machine learning model comprising an inference engine and a knowledge base, the machine learning model compares the associated first image against a patient data history and a tracking database;
in response to a determination the injury site is associated with the health condition, generating, by the one or more processors, a first result including sending a report containing the first result to a healthcare professional caring for the patient; and
displaying, by the one or more processors, the first result informing the patient and healthcare professional the injury site needs special care including requiring an appointment.

9. The computer system of claim 8, wherein the health condition comprises a diabetic foot and the patient data history comprises type of diabetes, blood glucose result, glycated hemoglobin result, and patient generated health data including daily blood glucose measurements, calories ingested, mood, and an amount of physical exercises done.

10. The computer system of claim 8, further comprising:
in response to a determination the injury site is not associated with the health condition, generating, by the one or more processors, a second result as a probability the injury site is associated with the health condition.

11. The computer system of claim 10, further comprising:
storing, by the one or more processors, the second result including the identifier associated with the patient, a date and time of the associated first image, the associated first image, and a report of an analysis in the tracking database.

12. The computer system of claim 10, further comprising:
in response to the second result displaying a normal result, generating, by the one or more processors, an alert comprising a reminder for the patient to maintain blood work levels associated with the health condition within a normal range.

13. The computer system of claim 8, further comprising:
prompting, by the one or more processors, the patient to take a second image of the injury site in a next day for tracking and evaluation;
in response to receiving the second image of the injury site, comparing, by the one or more processors, the second image with the associated first image using the machine learning model to determine whether a change occurred including whether the injury site is associated with the health condition; and
in response to a determination the injury site is not associated with the health condition while the second image includes changes, generating, by the one or more processors, a third result as a probability the injury site is associated with the health condition.

14. The computer system of claim 13, further comprising:
storing, by the one or more processors, the third result including updated clinical data of the patient and updated patient generated health data in the tracking database;
in response to the third result, displaying, by the one or more processors, a normal result comprising a current treatment and a reminder to maintain normal blood work levels; and
in response to a determination the wound is treated successfully, displaying, by the one or more processors, a health condition prevention result informing the patient and healthcare professional of a successful treatment including importance of maintaining normal blood work levels and returning for a medical appointment on a scheduled date.

15. A computer program product for a health condition accompanied by an injury site, comprising:
one or more computer readable storage media, and program instructions collectively stored on the one or more computer readable storage media, the program instructions comprising:
program instructions to receive, by one or more processors, an identifier associated with a patient and an associated first image, the first image corresponding to an injury in the injury site in the patient's body;
program instructions to analyze, by the one or more processors, the associated first image using a machine learning model comprising an inference engine and a knowledge base, the machine learning model compares the associated first image against a patient data history and a tracking database;
in response to a determination the injury site is associated with the health condition, program instructions to generate, by the one or more processors, a first result including sending a report containing the first result to a healthcare professional caring for the patient; and
program instructions to display, by the one or more processors, the first result informing the patient and healthcare professional the injury site needs special care including requiring an appointment.

16. The computer program product of claim 15, wherein the health condition comprises a diabetic foot and the patient data history comprises type of diabetes, blood glucose result, glycated hemoglobin result, and patient generated health data including daily blood glucose measurements, calories ingested, mood, and an amount of physical exercises done.

17. The computer program product of claim 15, further comprising:
in response to a determination the injury site is not associated with the health condition, program instructions to generate, by the one or more processors, a second result as a probability the injury site is associated with the health condition.

18. The computer program product of claim 17, further comprising:
program instructions to store, by the one or more processors, the second result including the identifier associated with the patient, a date and time of the associated first image, the associated first image, and a report of an analysis in the tracking database.

19. The computer program product of claim 17, further comprising:
in response to the second result displaying a normal result, program instructions to generate, by the one or more processors, an alert comprising a reminder for the patient to maintain blood work levels associated with the health condition within a normal range.

20. The computer program product of claim 15, further comprising:
program instructions to prompt, by the one or more processors, the patient to take a second image of the injury site in a next day for tracking and evaluation;
in response to receiving the second image of the injury site, program instructions to compare, by the one or more processors, the second image with the associated first image using the machine learning model to determine whether a change occurred including whether the injury site is associated with the health condition;
in response to a determination the injury site is not associated with the health condition while the second image includes changes, program instructions to generate, by the one or more processors, a third result as a probability the injury site is associated with the health condition;
program instructions to store, by the one or more processors, the third result including updated clinical data of the patient and updated patient generated health data in the tracking database;
in response to the third result, program instructions to display, by the one or more processors, a normal result comprising a current treatment and a reminder to maintain normal blood work levels; and
in response to a determination the wound is treated successfully, program instructions to display, by the one or more processors, a health condition prevention result informing the patient and healthcare professional of a successful treatment including importance of maintaining normal blood work levels and returning for a medical appointment on a scheduled date.
